(19) United States
(12) Patent Application Publication
(10) Pub. No.: US 2017/0145361 A1
(43) Pub. Date: May 25, 2017

(54) BIOREACTOR WITH SEPARATE CO2 SUPPLY

(71) Applicant: Subitec GmbH, Stuttgart (DE)

(72) Inventors: Peter Bergmann, Stuttgart (DE); Walter Troesch, Stuttgart (DE); Peter Ripplinger, Stuttgart (DE)

(21) Appl. No.: 15/419,384

(22) Filed: Jan. 30, 2017

Related U.S. Application Data
(63) Continuation-in-part of application No. PCT/EP2016/073473, filed on Sep. 30, 2016.

(30) Foreign Application Priority Data
Sep. 30, 2015 (GB) 1517290.1
Oct. 23, 2015 (GB) 1518810.5

(51) Int. Cl. C12M 1/00; C12M 1/36; C12M 1/04

(52) U.S. Cl. CPC C12M 21/02; C12M 23/34; C12M 23/24; C12M 41/48; C12M 23/58; C12M 29/24

(57) ABSTRACT

A bioreactor is disclosed. The bioreactor comprises at least one rise chamber containing a growth medium and at least one fall chamber connected to the at least one rise chamber at a top and at a bottom to form a loop. A first gas inlet is connected to the at least one rise chamber for supplying a gas flow to the at least one rise chamber and a second gas inlet supplied gas enriched with carbon dioxide.

### CROSS-REFERENCE TO RELATED APPLICATIONS

This application is a continuation-in-part of International Patent Application No. PCT/EP2016/073473 entitled “Bioreactor with interruptible gas supply” and filed on Sep. 30, 2016 and claiming benefit of and priority to UK Patent Application No 1517290.1 filed on Sep. 30, 2015 and UK Patent Application No. 1518810.5 filed on Oct. 23, 2015. All of these patent applications are incorporated herein by reference in their entirety.

### BACKGROUND OF THE INVENTION

Field of the Invention

The invention relates to a bioreactor for cultivating phototrophic microorganisms, such as but not limited to algae and cyanobacteria, as well as phototrophically grown cells, such as but not limited to moss. It furthermore relates to a method for operating such a bioreactor.

Brief Description of the Related Art

Bioreactors are fermenters in which a biological conversion of substances is carried out using, for example enzymes, microorganisms (bacteria, fungi, yeast, algae, cyanobacteria) as well as animal and plant cells, e.g. mosses. The operating conditions, such as temperature, pH value and nutrient condition can be adjusted within the bioreactor to optimize operating conditions for the conversion process. The function of the bioreactor include therefore transfer of materials in a fluid phase (mixing), dispersion in a second phase—in most cases air—in order to obtain a large phase boundary surface for good material transfer from gas to liquid, and thermal transfer for temperature regulation. The design of a bioreactor depends on its applications and therefore must consider the specific requirements of the biological system used.

In order to cultivate phototrophic microorganisms as well as phototrophically grown cells, so-called photobioreactors are used. Among potential designs, a so-called “airlift” photobioreactor can be used for cultivating phototrophic microorganisms and enabling the growth of the phototrophic microorganisms to a high cell density. Such airlift photobioreactors are produced by Subitec GmbH in Stuttgart.

The airlift loop photobioreactor has a column-shaped reactor vessel, in which introduction of air at the bottom of a rise chamber creates a fluid circulation of the growth medium within a loop defined by a shape of the photobioreactor. The airlift loop bioreactor is divided thus into a gassed zone (rise chamber) and an ungassed zone (down or fall chamber). The gassed zone and the ungassed zone are interconnected at their bottom and their top so that the hydrostatic pressure differential causes a pump effect that results in an overflow of the fluid containing the growth medium from the gassed zone. The mixing of the growth medium in the reactor is due substantially to the aeration volume per time and thus this photobioreactor design enables a good mixing and a high gas-fluid mass exchange (convective mass transfer) with low energy consumption. Such an airlift photobioreactor is described, for example, in the patent documents DE 199 16 597 or GB 2 235 210.

Another example of a photobioreactor is disclosed in U.S. Pat. No. 7,374,928 B2 (TrOsch), which discloses the presence of transversal septa (or dividing walls) arranged cross-wise to the airlift flow and constrict the airflow to form a meandering path (only the side view shows a meander)—within the photobioreactor. This meandering gas flow creates a turbulent liquid flow and thus promotes mixing of the phototrophic microorganisms and the phototrophically grown cells within the photobioreactor and creates light dilution for individual ones of the phototrophic microorganisms and the phototrophically grown cells, when the light source is unidirectional.

Other examples of photobioreactors are known in the art. For example European Patent Application No EP 2 840 128 teaches a photobioreactor which comprises a tank having an external wall, an inlet for adding a medium for algae growth, an outlet for extracting algae from the photobioreactor. The photobioreactor include a CO₂ applying means and lighting means. The tank has a first chamber and an intercommunicating second chamber, such that the medium of the tank can flow between the two chambers. The CO₂ applying means is arranged in the first chamber for supplying carbon dioxide to the medium and may also comprise means for applying a pressure wave. The pressure waves are described as “hitting” the carbon dioxide bubbles produced by the CO₂ applying means to increase the absorption of nutrients.

Chinese Utility Model CN 2015 93046 teaches also an air-lift circulating algae bioreactor. The bioreactor comprises a first pipe column and a second pipe column which are arranged at a distance from each other, an upper connecting pipe, a lower connecting pipe and an inflation unit. The first pipe column comprises a pipe wall defining a first incubating space, and the second pipe column comprises a pipe wall defining a second incubating space. An upper opening part and a lower opening part are formed at two opposite ends of each of the pipe walls. The upper and the lower connecting pipes are connected between the upper opening parts as well as between the lower opening parts, so as to communicate between the first and the second incubating spaces. The inflation unit can supply air or gas from an external gas source and provides a mechanism to force circulation between the first pipe column or the second pipe column, so that liquid filled therein can flow from one of the incubating spaces to the other incubating space to achieve the circulating flow effect.

US Patent Application Publication No US 2014/242581 teaches a photobioreactor system that may include one or more fluidly connectable tubes for growing or producing microorganisms or biomass, such as microalgae. The system may include either a single tube loop or an array of tubes. A first fluid may be held in the tube or tubes, and an inlet port may be provided for introducing a second fluid into the tube or tubes. The tube or tubes may be arranged vertically so that the second fluid rises through the tube or tubes. An outlet port may be provided at the top of the tube or tubes to remove the second fluid. The second fluid may be recirculated through the system via inlet and outlet lines as well as a pump and a replaceable reservoir for holding the second fluid. Where there is an array of tubes, a single inlet and outlet line may be sued for each tube.

International patent application No WO 2015/085155 teaches bioreactors that include a vessel with sides and a bottom. At least one opening in the vessel is connected to a means for introducing a gas, and at least one scaffold in the vessel is oriented substantially vertically in the vessel. The scaffolds are two substantially parallel sheets that are separated by a distance. Also disclosed are bioreactors that include a vessel with sides and a bottom, at least one opening in the vessel connected to a means for introducing a gas, and at least two scaffolds in the vessel oriented substantially vertically in the vessel. The patent application also includes methods of culturing cells including incubating a suspension of cells in a disclosed bioreactor and introducing a gas through the at least one opening in the vessel. In some examples, the cells include microalgae, macroalgae, bacteria, fungi, insect cells, plant cells, or animal cells.

Chinese Patent Application No 104328044 teaches a photobioreactor, which, during a photoautotrophic cultivation process, is used for adapting replenishment of carbon dioxide with light intensity by virtue of dynamic regulation of carbon dioxide by detecting changes of illumination intensity for photosynthesis and the concentration of liquid carbon dioxide in the system. The photobioreactor disclosed is used for effectively guaranteeing the coordination of energy and material input in the photosynthesis and is especially applicable to outdoor cultivation which uses natural light as a light source.

The prior art bioreactors all teach air or gas supply units which supply a stream of gas to one or more of the rise chambers bioreactors to achieve an “airlift” effect that mixes the growth medium, as described in the U.S. Pat. No. 7,374,928. This stream of gas includes carbon dioxide which is then used by the phototropic microorganisms for growth. The concentration of the carbon dioxide in the bioreactor decreases through the rise chamber and any unused carbon dioxide that has not dissolved into the growth medium or used by the phototropic microorganisms will be expelled through an exhaust valve.

### BRIEF SUMMARY OF THE INVENTION

This disclosure teaches a bioreactor comprising at least one rise chamber containing a growth medium and at least one fall chamber connected to the at least one rise chamber at a top and at a bottom to form a loop. A first gas inlet is connected to the at least one rise chamber for supplying a gas flow to the at least one rise chamber. The bioreactor includes further a second gas inlet connected in the loop which is ably to supply a second gas flow enriched with carbon dioxide. The second gas flow could be substantially pure carbon dioxide (up to 99%).

The use of two separate gas inlets enables one of the gas inlets to be used to generate the flow of the growth medium throughout the bioreactor and to enable turbulence to be generated at least within the rise chamber to promote growth of the phototropic organisms within the bioreactor. The second gas inlet is used to supply carbon dioxide in a controlled manner into the bioreactor.

The second gas inlet can be located at the top of the down chamber or at the bottom of the down chamber. There could indeed be more than one second gas inlet.

The second gas inlet has a plurality of porous openings to enable carbon dioxide to enter the growth medium in the down chamber and can be made of a plurality of injection needles or of a semi porous membrane.

The method of operation of a bioreactor comprises supplying the bioreactor with a growth medium for growing at least one of phototrophic microorganisms or phototrophically grown cells, supplying the bioreactor with a gas flow to intermix the growth medium, the growth medium including at least one of the phototrophic microorganisms or the phototrophically grown cells. The flow of the gas can be adjusted to optimize the growth by adjusting the interruption rate of the gas flow (leading to turbulence) to a given light intensity.

### BRIEF DESCRIPTION OF THE DRAWINGS

For a more complete understanding of the present invention and the advantages thereof, reference is now made to the following description and the accompanying drawings, in which:

FIG. 1 shows a bioreactor.

FIG. 2 shows a side view in form of a rise chamber of the bioreactor.

FIGS. 3A to 3F show a series of experimental results illustrating the interruption of the gas flow.

FIGS. 4A to 4C show of light and flow rate on the growth.

FIGS. 5A and 5B show a first arrangement of a carbon dioxide inlet located at the top of the down chamber.

FIG. 6 shows a second arrangement of a carbon dioxide nle ocated at the bottom of the down chamber.

FIGS. 7A and 7B show a plurality of injection needles used as the carbon dioxide inlet.

FIGS. 8A and 8B show in detail one of the injection needles in greater detail.

### DETAILED DESCRIPTION OF THE INVENTION

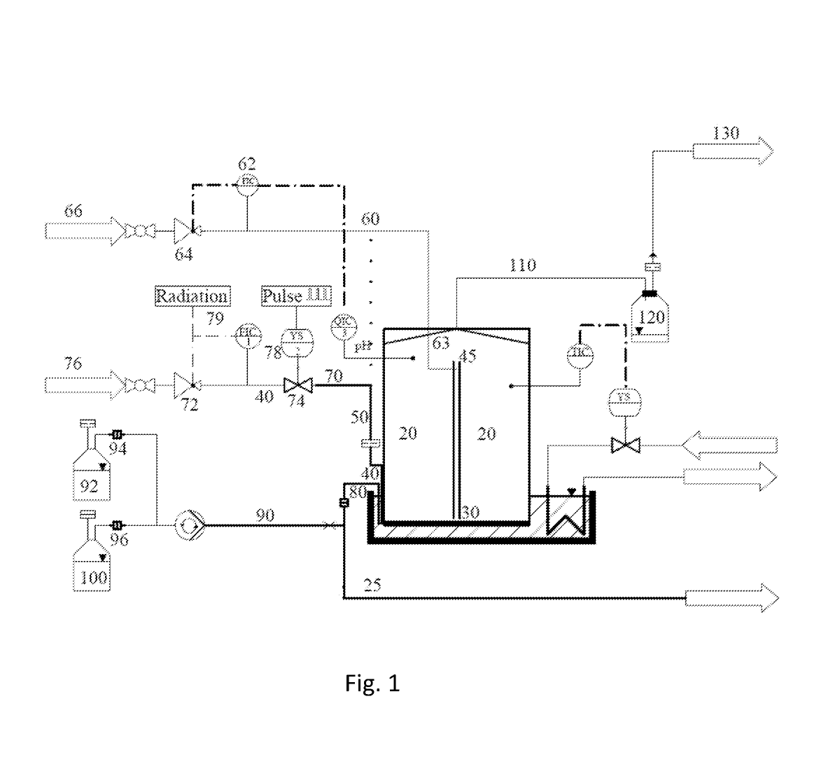

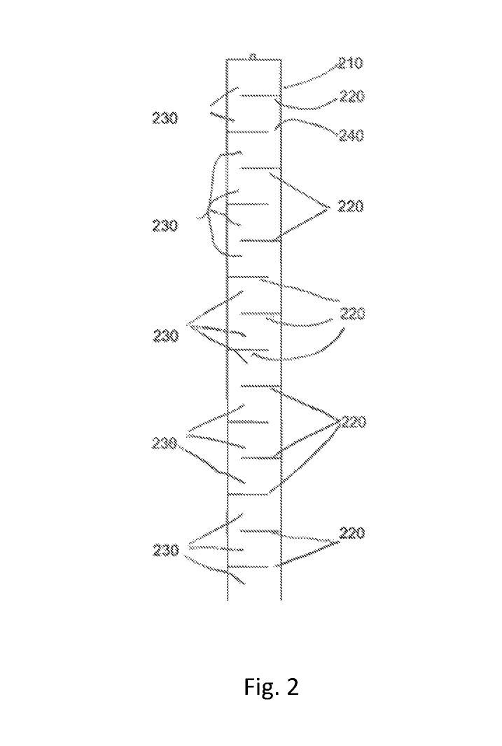

FIG. 1 shows a bioreactor 10 of the disclosure. The bioreactor 10 comprising two rise chambers 20 separated by a down chamber 30. The two rise chambers 20 and the down chamber 30 comprises a growth medium for growing phototrophic microorganisms, such as but not limited to algae or cyanobacteria, or phototrophically grown cells, such as mosses. Non-limiting examples of algae are the green algae Chlorella sorokiniana and the cyanobacterium Thermosynechococcus elongatus, The construction of the rise chambers 20 will be described in connection with FIG. 2: It will be appreciated that the bioreactor 10 of FIG. 1 has two rise chambers 20 and a single down chamber 30. Other bioreactors 10 may have a different number of rise chambers 20 and down chambers 30. The down chambers 30 can be arranged internally (as shown in FIG. 1) or external to the bioreactor 10.

The bioreactor 10 has a first gas inlet 40, which is connected to the bottom of the rise chambers 20 and carries gas from the gas pipe 50 and provides the gas to the rise chambers 20 to form a gassed zone within the rise chambers 20. The bioreactor 10 has also a medium input 80 connected to a medium pipe 90 which carries medium into the bioreactor 10. The bioreactor 10 has a second gas inlet 45 which is used to supply gas that is enriched with carbon dioxide to the bioreactor 10.

The second gas inlet 45 is connected to a carbon dioxide pipe 60, which carries carbon dioxide from a carbon dioxide source 66 through a carbon dioxide valve 64. The flow rate of the carbon dioxide in the carbon dioxide pipe is measured by the carbon dioxide flow meter 62.

The gas pipe 50 is further connected to an air pipe 70, which transports air from an air source, such as a compressor 76, through a mass flow controller 74 and/or a controllable magnetic air valve 72. The amount of air moving through the air pipe 70 can be controlled by a mass-flow controller 72. The magnetic air valve 72 and the mass flow controller 74 are connected to a controller 78, which measures and controls the flow of air through the air pipe 70, as will be described later. It will be appreciated that the magnetic air valve 72 could be replaced by other types of valves to open and close the air pipe 70 and thus control the movement of the air through the air pipe 70. It will also be appreciated that the air pipe 70 does not need both the magnetic air valve 72 and the mass flow controller 74. The mass flow controller 74 is particularly useful in connection with changing the flow of the air through the air pipe dependent on the amount of artificial light or sunlight falling on the bioreactor 10, as will be explained later.

The medium input 80 is connected to a medium pipe 90, which is connected through a first medium valve 94 to a first medium source 92 or through a second medium valve 96 to a second medium source 100. The first medium source 92 and the second medium source 100 have the various components of the growth medium for the biological material in the bioreactor 10.

The top of the bioreactor 10 has an outlet pipe 110 for removing excess gas through a filter 120 to an exhaust 130. The bioreactor 10 has at least one product outlet 25 to collect products produced in the bioreactor 10.

FIG. 2 shows an example of one of the rise chambers 20. The rise chamber has wall elements 210 made, in one aspect, of a thin, flexible, light-permeable plastic foil. The two wall elements 210 are constructed to have transverse elements 220 projecting from their inner sides. The transverse elements 220 are arranged parallel to each other and facing each other. In the displayed aspect of the bioreactor 10, there are seven transverse elements mounted on the inner sides of the wall elements 210. When the two wall elements 210 are placed together, as shown in FIG. 2, the transverse elements 220 form, in this example, fifteen interior chambers 230, divided by the transverse elements 220.

The transverse elements 220 do not completely close the interior of the rise chambers. There are still a plurality of small channels 240 formed between the inner wall of the wall elements 210 and the transverse elements 220. This plurality of small channels 240 allows passage of the medium and the gas from one end (bottom) of the rise chamber 20 through the interior chambers 230 to the top end of the rise chamber 20.

During operation of the bioreactor 10, the two rise chambers 20 are vertically positioned so that gas entering through the gas inlet 40 and the growth medium entering through the medium inlet 80 are allowed to rise through the small channels 240 and the interior chambers 230 of the rise chambers 20, as described in U.S. Pat. No. 7,374,928. The gas mixes the growth medium and supplies the growth medium with carbon dioxide. The plurality of transverse elements 220 thus form static mixers to enable mixing of the medium and the gas within the interior chambers 240. The gas is typically environmental air.

The gas is expelled through the outlet pipe 110. The growth medium is, as explained in U.S. Pat. No. 7,374,928, allowed to cycle down through the down chamber 30 where it is collected and allowed to rise again through the rise chambers 20.

The purpose of the first gas inlet 40 is to aerate the growth medium (to form convective mass transfer) by creating bubbles in the rise chambers 20 to mix up the growth medium, and thereby to mix up the phototrophic cells in the growth medium, drive the growth medium. The purpose of the second gas inlet 45 is to add carbon dioxide gas to the bioreactor 10 to enable growth. Typically, the air through the second gas inlet 45 is enriched to 1-10% by volume with scrubbed carbon dioxide gas, but could be substantially pure carbon dioxide (i.e. up to 99%). The use of substantially pure carbon dioxide gas leads to a large concentration gradient at the second gas inlet 45, which promotes absorption of the carbon dioxide gas by the growth medium.

Part of the carbon dioxide gas provided from the carbon dioxide source 66 will, however, be lost through the outlet pipe 110 with the expelled air. The aeration leads to elimination of any chemical concentration gradients in the bioreactor 10. It also addresses the effects of photolimitation and photoinhibition because of the growth of algae within the bioreactor 10 limiting the amount of light reaching the interior of the rise chambers 20 (so-called self shadowing due to the increasing phototrophic cell numbers per unit volume).

The second gas inlet 45 can be added to the top of or inside the down chamber 30 to add carbon dioxide at this point in the bioreactor 10. This separate second gas inlet 45 for the carbon dioxide would allow separate of the mixing and turbulence in the interior chambers by aeration from provision of the carbon dioxide supply. The separate inlet provides furthermore more time for the carbon dioxide in gaseous form to be absorbed into the medium during the cycle as the carbon dioxide would have time to be absorbed whilst the medium was being cycled down the down chamber 30, as well as up through the rise chamber 20. The carbon dioxide supply can be implemented substantially bubble free.

The air valve 72 and/or the mass flow controller 74 control the flow of the air from the compressor 76 through the bioreactor 10. The air valve 72 and/or the mass flow controller 74 works in conjunction with the controller 78 and can provide either a continuous flow of air (including in one aspect of the invention the carbon dioxide gas from the carbon dioxide pipe 60) to the bottom of the bioreactor 10 through the gas inlet 40 (as known in U.S. Pat. No. 7,374,928) or provide pulses of gas (including air and, if connected, carbon dioxide) by stopping and starting flow (“interrupting”) of the gas and thus creating discrete pulses of gas and not just a continuous gas flow. The provision of air through the air pipe 70 to the air inlet 40 consumes a significant amount of energy. By using the air valve 72 to provide the discrete pulses of air to the rise chambers 20 of the bioreactor 10, the amount of energy consumed by the bioreactor can be significantly reduced.

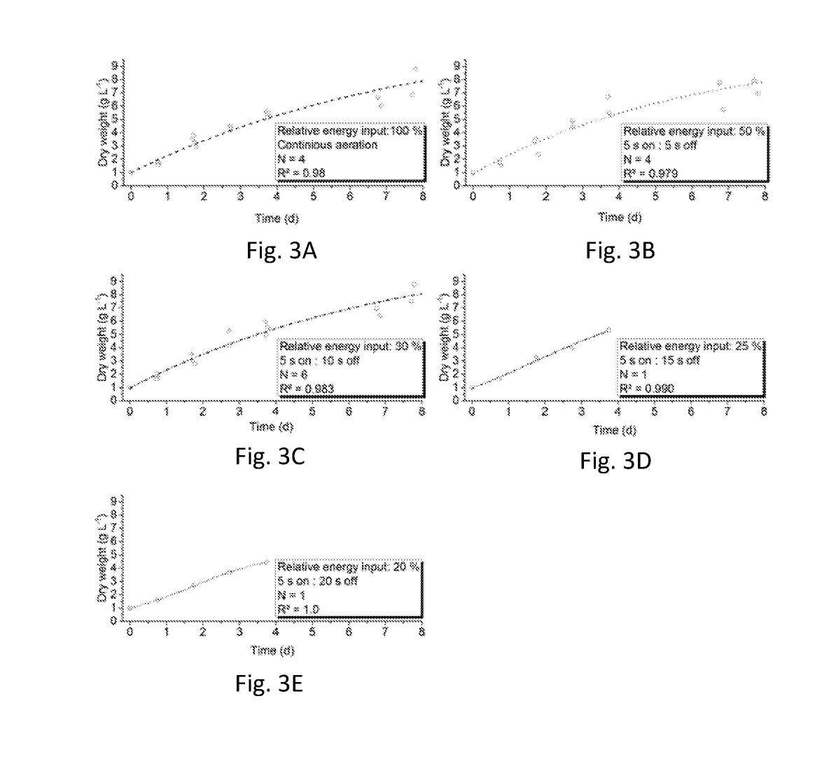

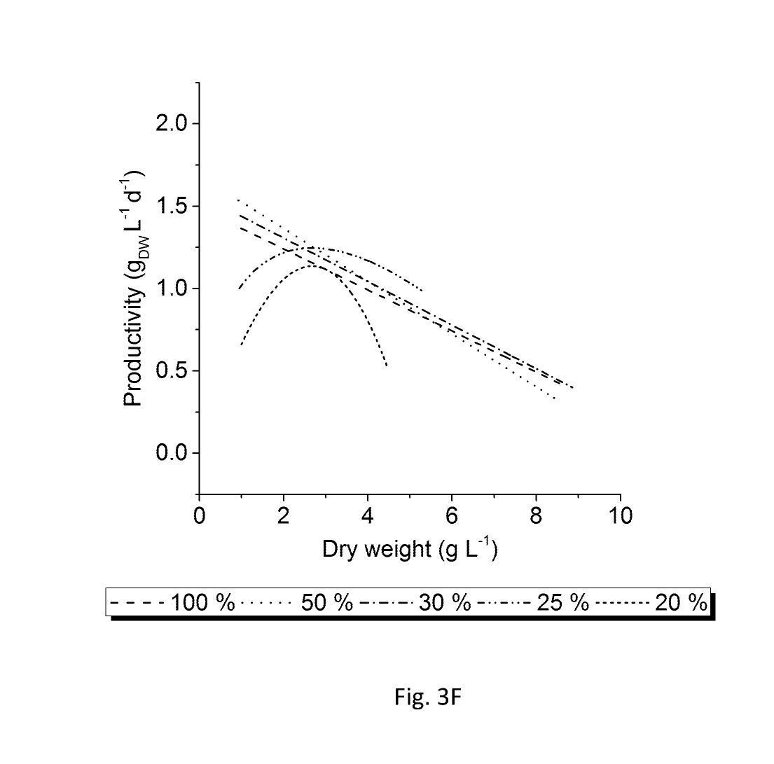

The graphs in FIGS. 3A-3E show the effect of providing the interruptible pulses of the air to the rise chambers for a 281 bioreactor 10. The first graph shows the growth of the material in the bioreactor over time in day for continuous air flow. Four tests were carried out (N=4) and the curve fitted with a standard deviation R² of 0.98. The second graph shows a 1:1 pulse rate for the air flow (in this case 5 s on and 5 s off) in which it will be seen that there is no significant amount of loss of productivity, but an energy saving of 50%. The third graph shows similar results for the case in which there is a 1:2 pulse rate, i.e. 5 s of a discrete block of pulsed air followed by a pause of 10 s. There is no significant loss of productivity, but only about ⅓ of the energy input.

The next two graphs show a loss of productivity, i.e. growth in the bioreactor 10 is substantially reduced and the air provided for turbulence is much smaller and not sufficient for optimal growth of the phototrophic cells. In both cases, the air was pulsed for five seconds and then turned off for 15 seconds or for 20 seconds. The experiments illustrated in FIGS. 3A-3E demonstrate that it is possible to reduce the amount of energy required to produce the air flow for optimal productivity for a given illumination.

These results are summarized in FIG. 3F which shows on the vertical axis the productivity (dry weight per liter of growth medium per day) and the dry weight per liter on the horizontal axis for various pulse rates. It will be seen that there is no loss of productivity between continuous flow and a 1:2 pulse rate, but that the productivity drops off for lower pulse rates for a given illumination.

The length of time of the discrete air pulses depends on the size of the reactor. The results shown in FIG. 3F are based upon a 28l reactor. A 180l reactor will require a longer pulse of air, but the relationship between the on and off of the discrete air pulses can be kept the same. Generally, it is thought that the discrete air pulse should be the same length as the time such that the air bubbles can rise from the bottom to the top through the rise chamber 20. It will be appreciated that the discrete air pulse leads to a formation of a series of air bubbles in the rise chamber 20.

In one aspect of the bioreactor 10, it is possible to adjust the air flow using the mass flow controller 74 in order to take into account the amount of light falling on the bioreactor 10 and detected by a PAR sensor 79. The PAR sensor 79 detects the photosynthetically active radiation (PAR) (similar but not identical to visible light) falling on the bioreactor 10. For example, on a cloudy day a significantly smaller amount of light, i.e. photosynthetically active radiation, will fall on the bioreactor 10, which means that the energy available for cultivation of phototrophic cells is reduced. This means that the amount of air required for mixing/dissipation the medium by aeration in the bioreactor 10 is reduced because of the lower light availability and slower growth. The controller 78 is connected to the PAR detector 79 and also to a flow meter and can measure and adjust the amount of air flowing through the air pipe 70 (and also the amount of carbon dioxide flowing through the carbon dioxide control valve 64 can be reduced). This will save energy and also reduce consumption of carbon dioxide by the bioreactor 10. For example, the amount of air flowing through the air pipe 70 will be adjusted to optimize the growth of the phototrophic cells in the bioreactor 10 dependent on the amount of artificial light or sunlight falling on the bioreactor 10.

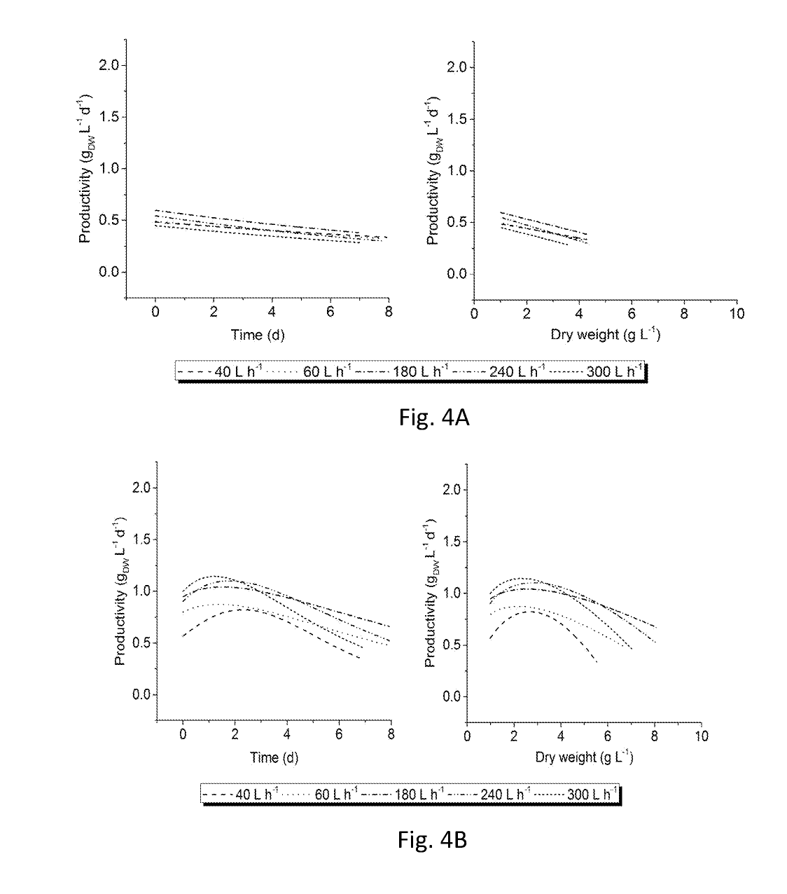

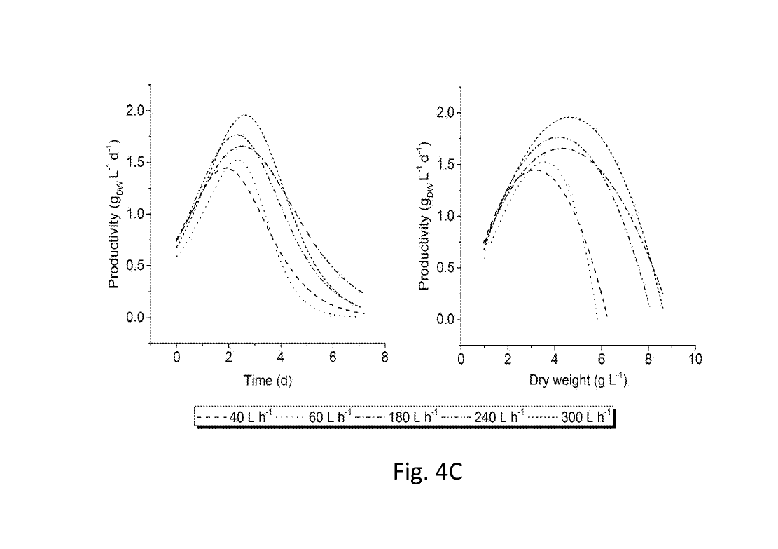

FIGS. 4A to 4C shows the effect of light on growth for various different differing flow rates of the air. FIG. 4A shows a light intensity of 180 μmol PAR photons/m²·s; FIG. 4B shows a light intensity of 405 μmol photons PAR/m²·s and FIG. 4C shows a light intensity of 780 μmol PAR photons/m²·s. It will be seen that, for the lower intensity light of FIG. 4A, the differing flow rates of the air has almost no effect on the growth rate in the bioreactor 10. The fall-off in the growth rate is due to the increasing concentration of the grown materials in the bioreactor 10 preventing use of some of the light. FIG. 4B shows a slight increase in the growth rate with increased flow rate of the air and FIG. 4C shows a much larger increase in the growth rate with increase flow rate of the air. Thus, one can conclude that at lower light intensities, the flow rate of the air can be reduced to save on energy without substantially affecting the productivity.

### EXAMPLES

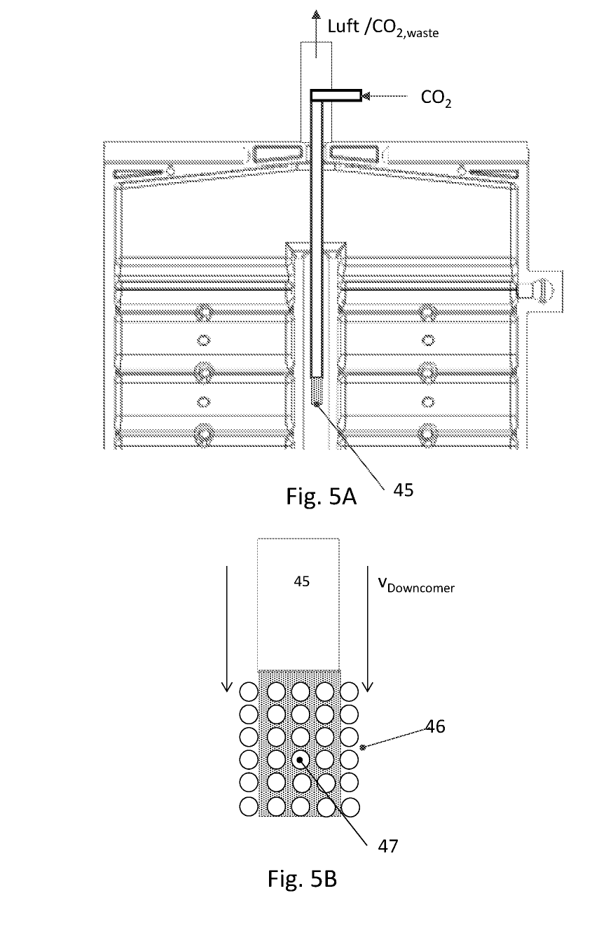

Different arrangements of the second gas inlet 45 will now be described. A first arrangement is shown in FIGS. 5A and 5B. The second gas inlet 45 is placed substantially in the centre of the down chamber of the bioreactor 10. The second gas inlet 45 has a head which is made of a semipermeable material or a porous membrane. Small bubbles 47 of the carbon dioxide gas appear on the surface of the head and are swept into the growth medium due to the circular flow of the growth medium in the bioreactor 10. The semipermeable material can be a ceramic, for example an oxide ceramic such as but not limited to an aluminium oxide. The semiporous membrane could be a polymer membrane, such as but not omitted to PES Polyethersulfon, PS Polysulfon and PPSU Polyphenylsulfon

It will be appreciated that in this arrangement, he second gas inlet 45 should be made with a substantially uniform pipe with no corner pieces, which might otherwise promote the growth of a film on the surface of the second gas inlet 45. The presence of the second gas inlet 45 in the down chamber 30 will also increase the flow at this position, because of the reduction in the cross-sectional area of the down chamber 30. This increase in flow also promote dissolution of the carbon dioxide in the growth medium.

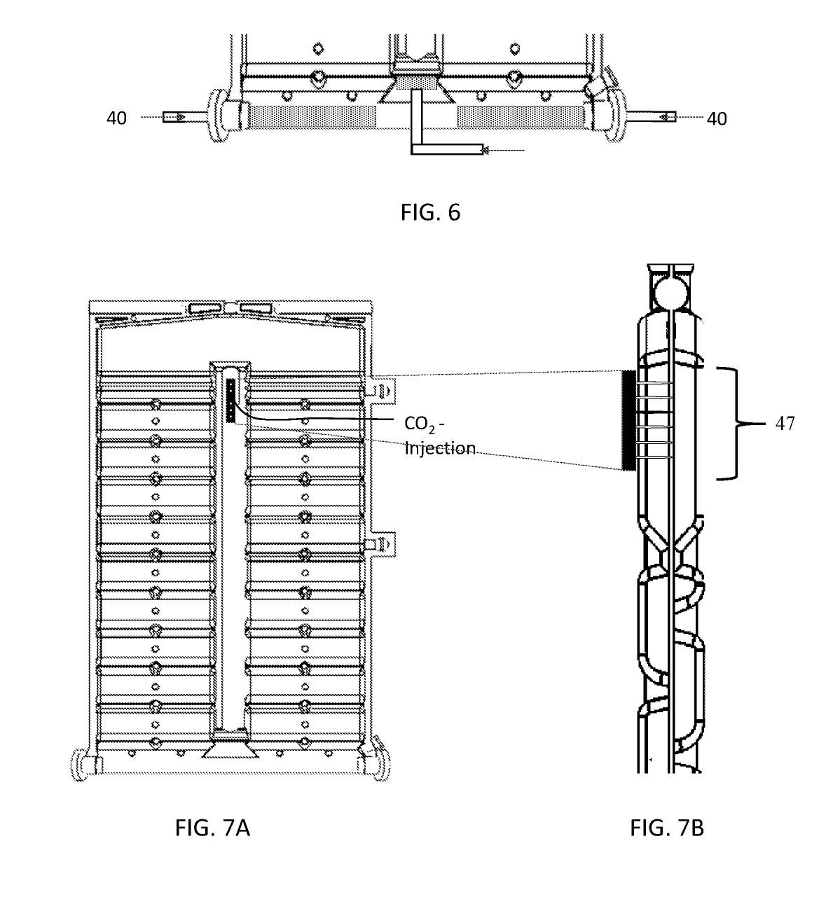

A second arrangement is shown in FIG. 6. The second gas inlet 45 is located at the bottom of the down chamber. There will be an increased hydrostatic pressure on the second gas inlet 45, but this can be compensated by an increased gas pressure at the second gas inlet 45. The area through which the carbon dioxide gas can enter the growth medium is increased due to the larger possible area of the porous membrane.

This second arrangement can be retrofitted to existing ones of the bioreactors 10. The location adjacent of the second gas inlet 45 substantially adjacent to the first gas inlet 45 means that the turbulent flow generated in the rise chamber 20 promotes the dissolution of the carbon dioxide gas into the growth medium.

A third arrangement is like the first arrangement in that the second gas inlet 45 is located at the top of the down chamber 30. However, a plurality of injection ports 47 are used to inject the carbon dioxide gas into the down chamber 30. This third arrangement with the plurality of injection ports 47 is shown in FIGS. 7A and 7B.

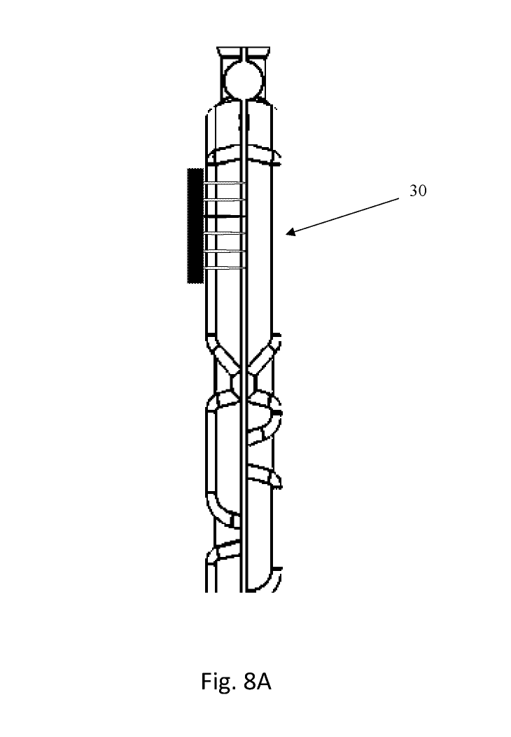

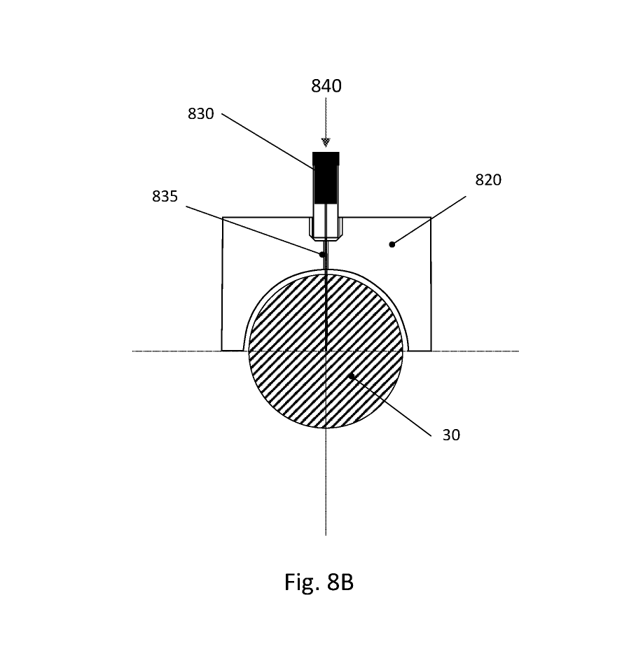

The plurality of injection ports 47 are shown in more detail in FIG. 8A (side view) and FIG. 8B (cross-section). The injection ports 47 enable more control of the flow rate. The small size of the injection needles 47 do not offer much resistance to the flow of the growth medium in the down chamber. The number of the injection needs 47 can be increased to increase the amount of carbon dioxide injected into the growth medium. It will be appreciated that this third arrangement can also be retrofitted to exiting bioreactors 10.

The construction of the injection ports 47 can be seen in FIG. 8B in which an injection needle 810 is present in the down chamber 30. The injection needle 810 is mounted in a half block 820 made, for example of aluminum, about the down chamber 30. An injection canal 830 is located within the half block 820 and connects inlet 840 with a bored hole 835, which is in turn connected to the injection needle 810.

### REFERENCE NUMERALS

10 Bioreactor
20 Rise chamber
25 Product outlet
30 Down chamber
40 First gas inlet
45 Second gas inlet
46 Membrane
47 Bubbles
50 Gas pipe
60 Carbon dioxide pipe.
62 Flowmeter
64 Carbon dioxide valve
66 Carbon dioxide source
70 Air pipe
72 Air valve
74 Mass-flow controller
76 Compressor
78 Controller
79 PAR sensor
80 Medium inlet
90 Medium pipe
92 First medium source
94 First medium valve
96 Second medium valve
100 Second medium source
110 Outlet pipe
210 Wall elements
220 Transverse elements
230 Interior chambers
240 Channel
810 Injection needle
820 Half block
830 Injection Canal
835 Bored hole
840 Inlet

What is claimed is:

1. A bioreactor comprising:
at least one rise chamber containing a growth medium;
at least one down chamber connected to the at least one rise chamber at a top and at a bottom to form a loop;
a first gas inlet connected to the at least rise chamber for supplying a first air flow to the at least one rise chamber; and
a second gas inlet connected in the loop to supply a second gas flow to the loop, the second gas flow being enriched, compared to air, with carbon dioxide.

2. The bioreactor of claim 1, wherein the second gas flow is substantially pure carbon dioxide.

3. The bioreactor of claim 1, wherein the second gas inlet is located in the at least one down chamber.

4. The bioreactor of claim 1, wherein the second gas is substantially bubble free in the growth medium.

5. The bioreactor of claim 1, wherein the second gas inlet is located at the boom of the at least one down chamber.

6. The bioreactor of claim 1, wherein the second gas inlet is located at the top of the at least one down chamber.

7. The bioreactor of claim 1, wherein the second gas inlet comprises a plurality of porous openings in the at least one fall chamber.

8. The bioreactor of claim 1, wherein the porous openings are formed of at least one of a plurality of injection needles or a semiporous membrane.

9. The bioreactor of claim 1, further comprising a controller for controlling the flow rate of the second gas flow.

10. The bioreactor of claim 1, wherein the growth medium is used for growing phototrophic microorganisms or phototrophically grown cells.

11. The bioreactor of claim 10, wherein the phototrophic microorganisms are at least one of algae or cyanobacteria.

12. A method of operation of a bioreactor comprising:
supplying the bioreactor with a growth medium for growing at least one of phototrophic microorganisms or phototrophically grown cells;
supplying the bioreactor with a first gas flow to intermix the growth medium, the growth medium including at least one of the phototrophic microorganisms or the phototrophically grown cells; and
supplying the bioreactor with a second gas flow, the second gas floe being, compared to air, enriched with carbon dioxide.

13. The method of operation of claim 12, further comprising adjustment of the second gas flow dependent on concentration of the carbon dioxide in at least one of the growth medium or exhaust gas.
